(19) United States
(12) Patent Application Publication
(10) Pub. No.: US 2014/0364785 A1
(43) Pub. Date: Dec. 11, 2014

(54) APPARATUS FOR STABILIZATION OF PELVIC FRACTURES

(71) Applicant: John Moore, Anaconda, MT (US)

(72) Inventors: John E. Moore, Anaconda, MT (US); Joel Switzer, Bozeman, MT (US); David Yakos, Bozeman, MT (US)

(21) Appl. No.: 13/913,488

(22) Filed: Jun. 9, 2013

(51) Int. Cl. A61F 5/058

(52) U.S. Cl. CPC A61F 5/058
USPC 602/19

(57) ABSTRACT

An apparatus for stabilization of pelvic fractures comprising a stationary pad member, an adjustable pad member, and two trochanter pads. The stationary pas member comprises a cross-member with a rack gear. The adjustable pad assembly is configured to slide laterally on the cross-member so that the distance between the first and second trochanter pads is adjustable. The invention further comprises a torque release clutch that is configured to cause the retaining pawl gear tooth to disengage from the rack gear teeth when a certain force is applied to the torque release clutch.

### BACKGROUND OF THE INVENTION

1. Field of the Invention

The present invention related generally to the field of medical devices and, more specifically, to an apparatus for stabilization of pelvic fractures.

2. Description of the Related Art

The use of pelvic splints is well known in prior art. There are a number of sling devices where the primary purpose is stabilization of suspected pelvic fractures by application of circumferential pressure. Although these devices largely fulfill their objectives, there remains a need for a device that protects against over-tightening and is more user-friendly in its application. Existing art requires that the patient be lifted, which often requires two operators. The sling is then placed around the pelvis is posteriorly by a third operator and grasped and pulled around the pelvis circumferentially. It is then tightened by the third operator. Some devices require that more than one strap be tightened. Patients with unknown spinal injuries are placed at abject risk for further injury by this untoward mobilization.

Typical construction of these prior art devices consists of a soft band of foam covered in cloth that is wrapped around the pelvis with no constraints as to how loosely or tightly it is bound. The present invention overcomes many of the shortcomings of prior art devices by requiring less than circumferential application. This allows for quick placement by a single operator, which is critical in pre-hospital and emergency room setting, and it limits operator error and the inherent risk of injury to the patient by preventing over-tightening. The present invention allows the weight of the patient to provide posterior stabilization of the pelvis while providing lateral stabilization of the pelvis. The circumferential compression required by prior art devices is time-consuming and inefficient in situations where minutes and even seconds matter.

Examples of prior art devices liar hip and/or pelvic stabilization include U.S. Patent Application Pub. No. 2012/0209162 (Bar-Natan et al.) (circumferential pelvic splint with a ratchet mechanism); U.S. Pat. No. 6,626,856 (Manoach, 2003) (circumferential semi-rigid pelvic compression splint); and. U.S. Pat. No. 6,500,137 (Molino et al., 2002) (circumferential pelvic region orthotic device); see also U.S. Pat. No. 5,437,618 (Sikes, 1995) (joint protection device comprised of a rigid outer shell and at least one cushioned pad on its inner face). As explained more fully below, none of these prior art devices comprises the structural features of the present invention.

### BRIEF SUMMARY OF THE INVENTION

The present invention is an apparatus for stabilization of pelvic fractures comprising: a stationary pad assembly comprising a stationary pad member and a first trochanter pad, the stationary pad member comprising a cross-member, and the cross-member comprising a rack gear with rack gear teeth; an adjustable pad assembly comprising arm adjustable pad member and a second trochanter pad; wherein there is a distance between the first trochanter pad and the second trochanter pad, and wherein the adjustable pad assembly is configured to slide laterally on the cross-member so that the distance between the first trochanter pad and the second trochanter pad is adjustable. Preferably, each of the first and second trochanter pads is configured to fit over a greater trochanter of a human.

In a preferred embodiment, the adjustable pad member comprises a handle, an advancing pawl, and a retaining pawl; wherein the cross-member fits into cross-member apertures in the adjustable pad member; wherein the handle, advancing pawl and retaining pawl are situated in a handle recess in front of the cross-member apertures; wherein the handle comprises a first lateral arm and a second lateral arm; wherein an advancing pawl pin extends through a central axis of the advancing pawl, through a first end of the first lateral arm and into a first advancing pawl pin hole in a front end of the adjustable pad assembly and through a first end of the second lateral art and into a second advancing pawl pin hole in the front end of the adjustable pad assembly; wherein a returning pawl pin extends through a retaining pawl pin hole in the retaining pawl and into a first retaining pawl pin hole in the front end of the adjustable pad assembly and a second retaining pawl pin hole in the front end of the adjustable pad assembly; wherein the retaining pawl comprises a button and a retaining pawl gear tooth; wherein when the button is pushed by an operator, the retaining pawl gear tooth disengages from the rack gear teeth of the rack gear, thereby allowing the adjustable pad member to slide laterally on the rack gear, and wherein when the retaining pawl gear tooth is engaged with the rack gear teeth, the adjustable pad member is prevented from sliding laterally on the rack gear. Preferably, the invention further comprises a retaining pawl spring that is configured to bias the retaining pawl gear tooth toward engaging with the rack gear teeth.

In this preferred embodiment, extension of the handle causes the advancing pawl to rotate, and rotation of the advancing pawl causes the retaining pawl gear tooth to disengage from the rack gear teeth until the retaining pawl spring forces the retaining pawl gear tooth to reengage with the rack gear teeth.

In another preferred embodiment, the adjustable pad member comprises a knob and a retaining pawl; wherein the knob is connected to a pinion gear in pinion shaft; wherein the pinion gear comprises pinion teeth; wherein the retaining pawl comprises a release slide that is attached via a release slide bracket to a support member that comprises a retaining pawl gear tooth; wherein the retaining pawl further comprises a retaining pawl pin that extends in a forward direction from the support member and is inserted into a retaining pawl hole in a front portion of the adjustable pad member; wherein the release slide bracket extends through a release slide slot in the front portion of the adjustable pad member; wherein the pinion gear shaft extends through an advancing knob hole in the front portion of the adjustable pad member; wherein the retaining pawl comprises a retaining pawl gear tooth; wherein when the release slide is moved vertically within the release slot, the retaining pawl gear tooth disengages from the rack gear teeth of the rack gear, thereby allowing the adjustable pad member to slide laterally on the rack gear; and wherein when the retaining pawl gear tooth is engaged with the rack gear teeth, the adjustable pad member is prevented from sliding laterally on the rack gear. Preferably, the invention further comprises a retaining pawl spring that is configured to bias the retaining pawl gear tooth toward engaging with the rack gear teeth.

In this preferred embodiment, rotation of the knob causes the pinion gear to rotate, and rotation of the pinion gear causes the retaining pawl gear tooth to disengage from the rack gear teeth until the retaining pawl spring forces the retaining pawl gear tooth to reengage with the rack gear teeth.

In a preferred embodiment, the adjustable pad assembly comprises a retaining pawl with a retaining pawl gear tooth, and the invention further comprises a torque release clutch that is configured to cause the retaining pawl gear tooth to disengage from the rack gear teeth when a certain force is applied to the torque release clutch.

### BRIEF DECRYPTION OF THE DRAWINGS

FIG. 1 is a perspective view of the present invention.

FIG. 2 is a top view of the present invention at minimum pad distance.

FIG. 3 is a top view of the present invention at maximum pad distance.

FIG. 4 is a perspective view of a patient on a backboard with the present invention in use.

FIG. 5 is a front view of the present invention on a torso showing pad alignment to the greater trochanters and cross-member alignment to the public symphysis.

FIG. 6 is a top view of the present invention on a torso showing pad alignment to the greater trochanters and cross-member alignment to the pubic symphysis.

FIG. 7 is a right side view of the present invention on a torso showing pad alignment to the greater trochanters and cross-member alignment to the pubic symphysis.

FIG. 8 is an exploded view of the present invention.

FIG. 9 is a perspective view of the retaining pawl assembly.

FIG. 10 is a perspective view of the retaining pawl assembly.

FIG. 11 is an exploded view of the advancing lever assembly.

FIG. 12 is an exploded view of the retaining pawl assembly.

FIG. 13 is a front detail view of the adjustable pad assembly.

FIG. 14 a top cross-section view of the ratchet in a neutral position.

FIG. 15 is a top cross-section less of the ratchet in an advancing position.

FIG. 16 is a top cross-section view of the ratchet in an advanced position.

FIG. 17 is a top cross-section view of the ratchet in a releasing position.

FIG. 18 is a detail view of the torque release clutch in an engaged position.

FIG. 19 is a detail view of the torque release clutch in a disengaged position.

FIG. 20 is a detail view of an alternate closure and release system.

FIG. 21 is an exploded view of the alternate closure and release system.

FIG. 22 is a from cross-section view of the alternate closure and release system in a neutral or advanced position.

FIG. 23 is a front cross-section view of the alternate closure and release system in an advancing position.

FIG. 24 is a front cross-section view of the alternate closure and release system in a released position.

FIG. 25 is an exploded view of the knob assembly.

FIG. 26 is a front cross-section view of an alternate torque release clutch in an engaged position.

FIG. 27 is a front cross-section view of an alternate torque release clutch in a disengaged position.

FIG. 28 is a side cross-section view of the embodiment shown in FIG. 25.

FIG. 29 is a perspective view of the adjustable pad member 16 showing the attachment point for the spring 16D.

FIG. 30 is a perspective view of an alternate embodiment of the stationary pad member.

### REFERENCE NUMBERS

1 Pelvic brace assembly
2 Stationary pad assembly
3 Stationary pad member
3B Cross-member
3C Rack gear
3D Rack gear Tooth
3E Rack gear recess
4 Trochanter pad
5 Adjustable pad assembly
6 Adjustable pad member
6A Cross-member aperture
6B Handle recess
6C Advancing pawl pin hole
6D Retaining pawl pin bole
6E Retaining pawl spring
7 Advancing lever assembly
8 Handle
8A Torque release clutch (first face)
8B Advancing pawl in hole (in handle)
8C Lateral arm (of handle)
9 Advancing pawl pin
10 Advancing pawl
10A Advancing pawl goat tooth
10B Non-toothed section
10C Torque release clutch (second face)
10D Advancing pawl pin hole
11 Retaining pawl assembly
12 Retaining pawl pin
13 Retaining pawl
13A Retaining pawl pin hole
13B Retaining pawl gear tooth
13D Button
14 Human
14A Pelvis
14B Femur
14C Iliac crest
14D Greater trochanter
14E Public symphysis
14F Sacrum
14G Sacroiliac joint
14H Epidermis
15 Backboard
16 Adjustable pad member (alternate embodiment to reference number 6)
16A Advancing knob hole
16B Retaining pawl hole
16C Release slide slot
16D Retaining pawl spring
17 Advancing knob assembly (alternate embodiment ti advancing lever assembly)
17A Knob
17B Snap fit attachment (female)
17C Pinion gear
17D Inner torque release clutch
17E Outer torque release clutch
17F Snap fit attachment (male)
17G Pinion gear tooth
17H Pinion gear shaft
18 Retaining pawl (alternate embodiment)
18A Release slide
18B Retaining pawl pin
18C Support member
18D Release slide bracket
18E First end (of retaining pawl)
19 Stationary pad assembly (alternate embodiment to reference number 2)
19A Stationary pad member
19B Cross-member
19C Rack gear
19D Rack teeth
19 E Rack gear recess

### DETAILED DESCRIPTION OF INVENTION

The present invention has many advantages over the prior art in terms of the reduction and stabilization of open book pelvic fractures. The present invention is a pelvic stabilizer that requires less than circumferential application, which means that fewer operators are required to install the device, the device can be installed more quickly, and further injury to the patient can be minimized because the patient does not need to be lifted. In addition, the present invention is designed to prevent over-tightening of the device. To install the present invention on a patient, a single operator places the device in an open position over the patient's pelvis, aligning it with the pubic symphysis, and then tightens it to a preset amount of pressure, thereby allowing the device to fit snugly and securely around the patient.

The present invention generally comprises a rigid cross-member and two lateral stabilization pads. One of the pads is stationary, and the other moves laterally to increase or decrease the pressure on the patient. The invention is designed to provide physical limitations on the degree to which the one pad may be moved toward or away from the other pad, primarily by incorporating a torque release clutch. The invention thus limits over-tightening and minimizes the potential for co-morbidities associated with pelvic fractures, such as hemorrhage or damage to organs and soft tissue structures, as well as secondary injury to the surface of the skin, including dermabrasion and pressure necrosis. Because the apparatus is tightened to the appropriate force, it avoid over-compression of the pelvis, which itself can lead to increase morbidity and mortality.

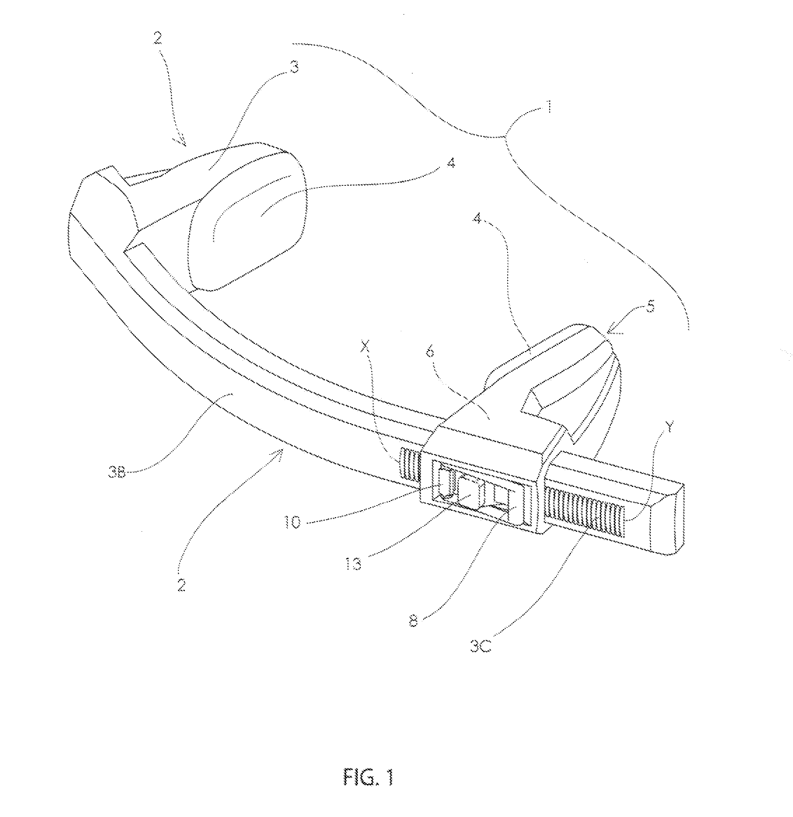

FIG. 1 is a perspective view of the present invention. As shown in this figure, the pelvic brace assembly 1 comprises a stationary pad assembly 2 and an adjustable pad assembly 5. The stationary pad assembly 2 comprises the stationary pad member 3 and the trochanter pad 4. The stationary pad member 3 comprises a cross-member 3B, which in turn comprises a rack gear 3C. A trochanter pad 4 is attached to the stationary pad member 3. The adjustable pad assembly 5 is designed to move laterally along the rack gear 3C portion of the cross-member 3B. The adjustable pad assembly 5 comprises the adjustable pad member 6 and the trochanter pad 4. This figure also shows the handle 8, advancing pawl 10 and retaining pawl 13. A trochanter pad 4 is attached to the adjustable pad member 6.

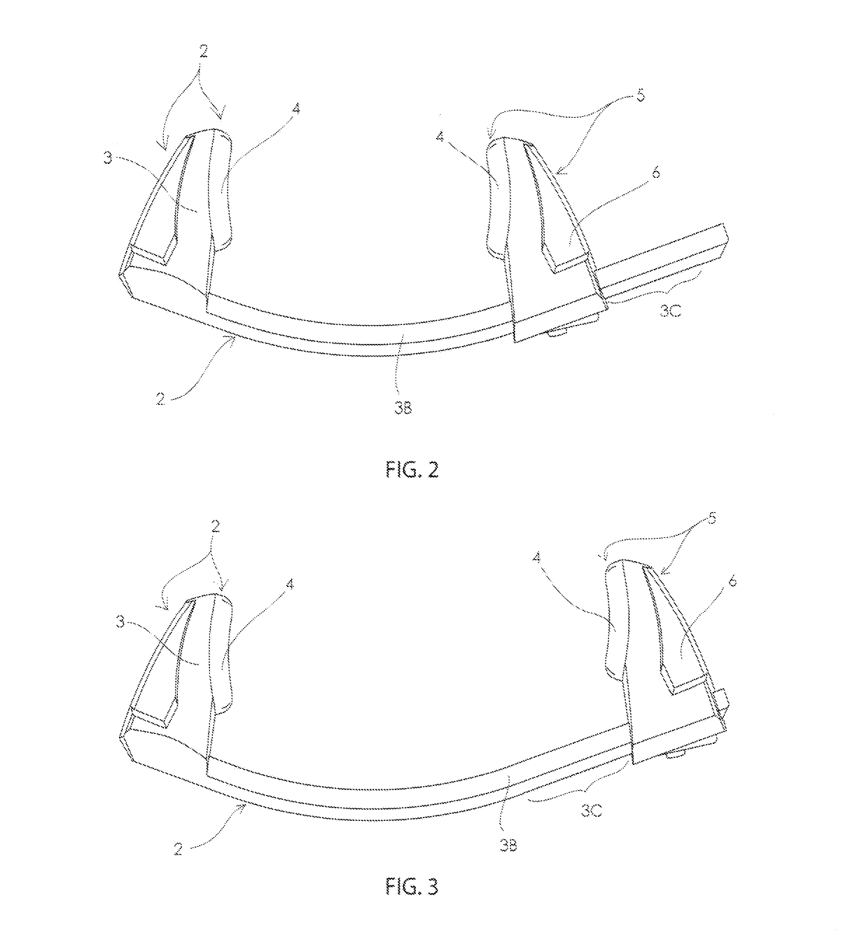

FIG. 2 is a top view of the present invention at minimum pad distance. As illustrated in FIG. 2, the lateral movement of the adjustable pad assembly 5 along the cross-member 3B of the stationary pad assembly 2 is limited by the length of the rack gear 3C. In other words, the adjustable pad assembly 5 can only move as close to the trochanter pad 4 on the stationary pad member 3 as the proximal end (designated with an “X” on FIG. 1) of the rack gear 3C and as far away from the trochanter pad 4 on the stationary pad member 3 as the distal end (designated with a “Y” FIG. 1) of the rack gear 3C. FIG. 3 is a top view of the present invention at maximum pad distance.

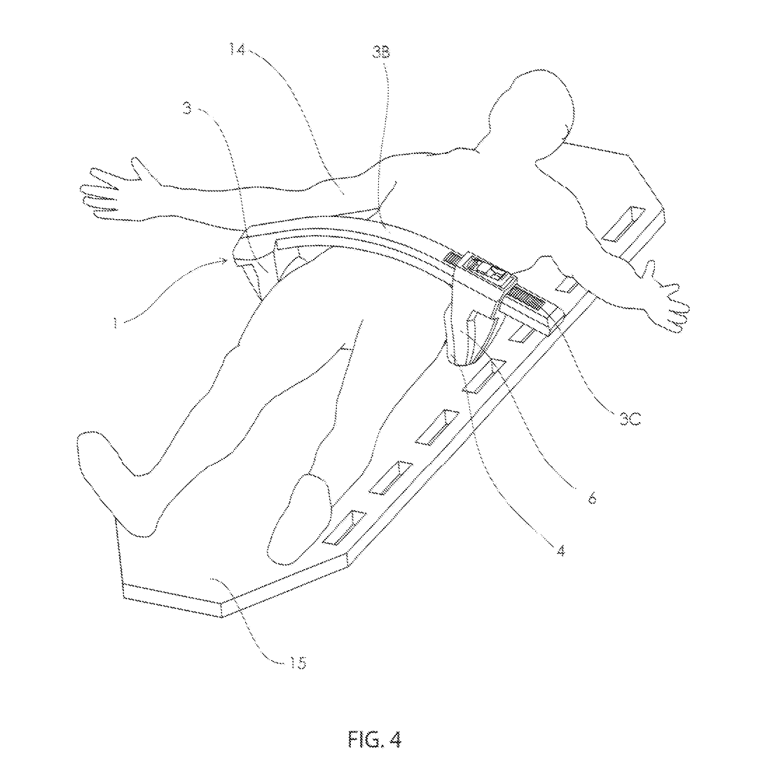

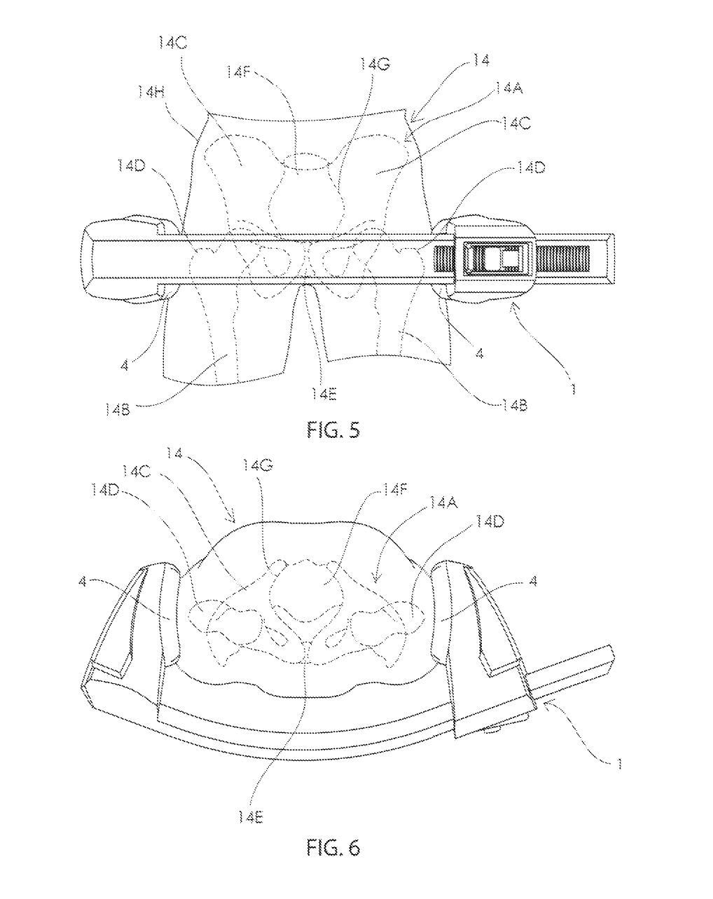

FIG. 4 is a perspective view of a patient on a backboard with the present invention in use. To install the apparatus on a patient, the adjustable pad assembly 5 is slid to the distal end of the rack gear 3C, and the cross-member 3C is positioned over the patient's abdomen and the adjustable pad assembly 5 is slid toward the proximal end of the rack gear 3C until the trochanter pads 4 are situated adjacent to the patient's right and left greater trochanters (see FIG. 5). The length of the rack gear 3C limits the inward movement of the adjustable pad assembly 5. By virtue of the fact that the distance between the two trochanter pads 4 is adjustable, the apparatus may be used on patients of different sizes.

FIG. 5 is a from view of the present invention on a torso showing pad alignment to the greater trochanters and cross-member alignment to the pubic symphysis. As shown in this figure the two trochanter pads 4 are preferably aligned so that they are directly adjacent to the patient's right and left greater trochanters 14D. FIG. 6 is a top view of the present invention on a torso showing pad alignment to the greater trochanters and cross-member alignment to the pubic symphysis.

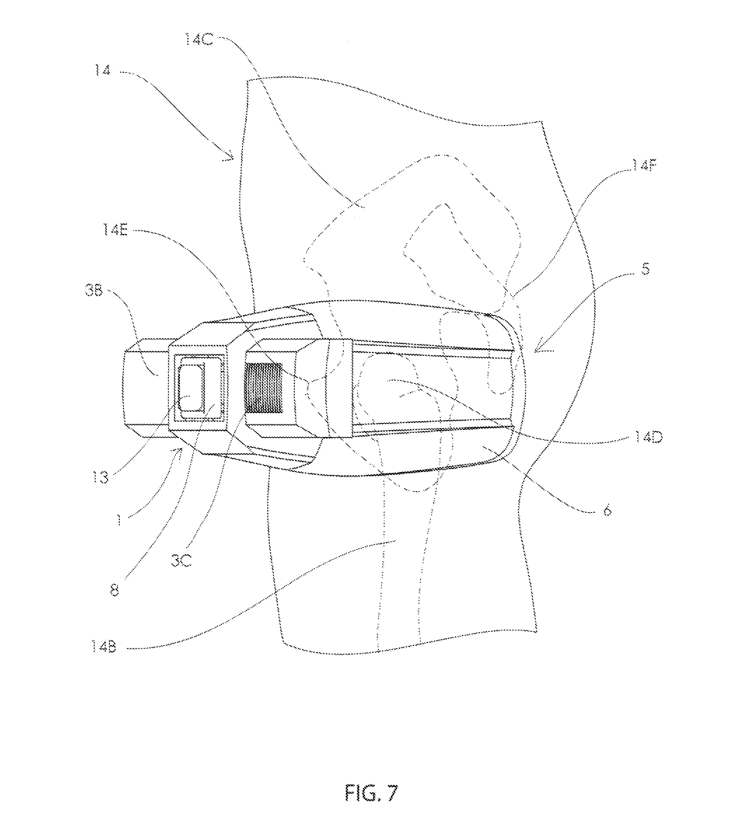

FIG. 7 is a right aide view of the present invention on a torso showing pad alignment to the greater trochanters and cross-member alignment to the pubic symphysis.

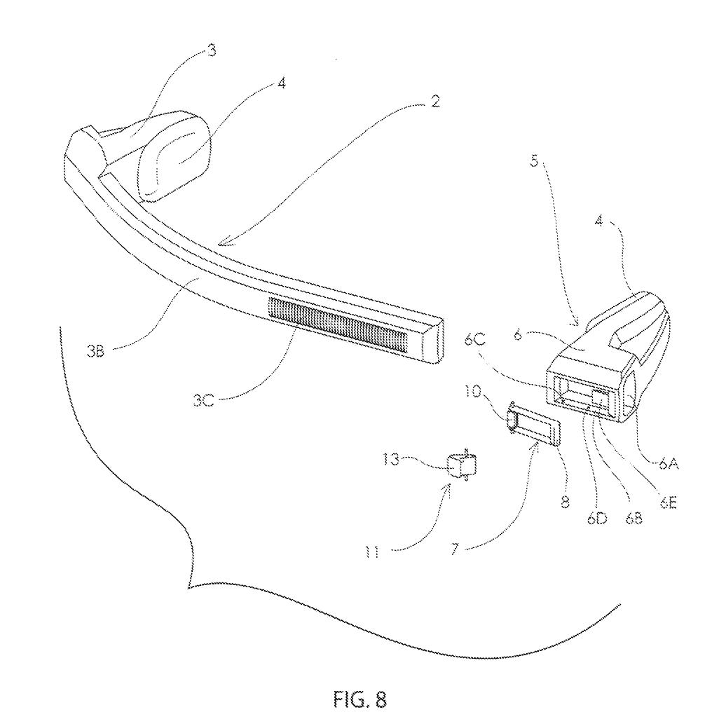

FIG. 8 is an exploded view of the present invention. This figure shows the stationary pad assembly 2 and the adjustable pad assembly 5. The adjustable pad assembly 5 comprises an adjustable pad member 6. The cross-member 3B is into cross-member apertures 6A (or windows) on either side of the adjustable pad member 6. The advancing lever assembly 7, which includes the handle 8 and the advancing pawl 10, and the retaining pawl assembly 11, which includes the retaining pawl 13, are situated in the handle recess 6B at the front of the adjustable pad member 6. The end the cross-member 3B that includes the rack gear 3C is inserted laterally into the cross-member aperture 6A, as shown in FIG. 1.

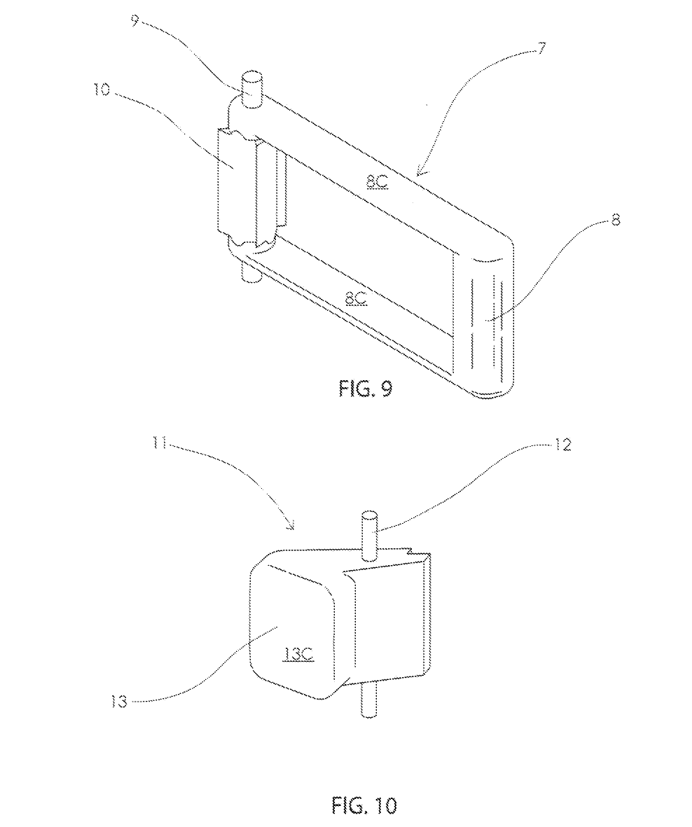

FIG. 9 is a perspective view of the advancing lever assembly. As shown in this figure, the handle 8 comprises two lateral sons 8C. An advancing pawl pin 9 extends through the ends of the two lateral arms 8C and also through the advancing pawl 10.

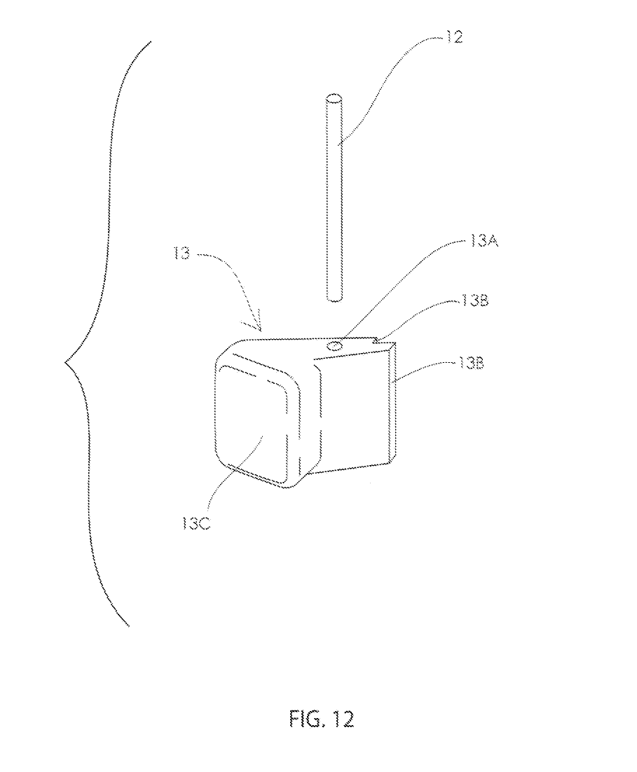

FIG. 10 is a perspective view of the retaining pawl assembly. As shown in this figure, the retaining pawl assembly 11 comprises a retaining pawl 13. A retaining pawl pin 12 extends through a retaining pawl pin hole 13A (see FIG. 12) in the retaining pawl 13. The retaining pawl 13 comprises a button 13C.

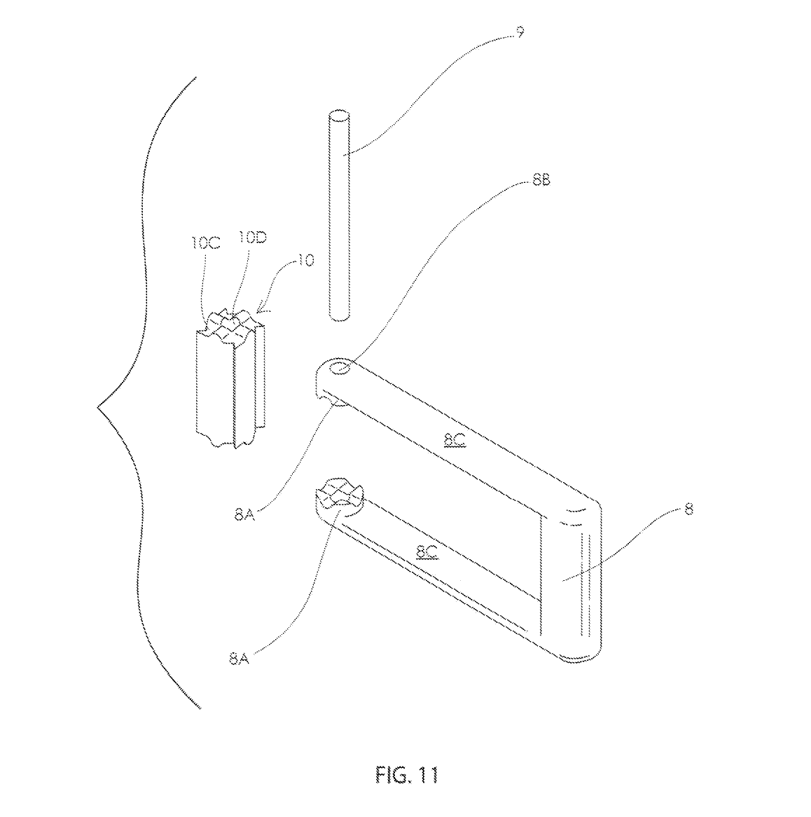

FIG. 11 is an exploded view of the advancing lever assembly. The advancing pawl pin 9 extends through an advancing pawl pin hole 8B in each lateral arm 8C of the handle 8 and also through an advancing pawl pin hole 10D in the advancing pawl 10. As shown in this figure, the advancing pawl pin hole 8B extends through a first face 8A of the torque release clutch on the inside surface of each of the two lateral arms 8C. On either end of the advancing pawl 10 is a second face 10C of the torque release clutch. (In other words, the first face 8A and the second face 10C together comprise the torque release clutch.) When the retaining pawl assembly 11 is installed, the first face 8A and second thee 10C of the torque release clutch fit together as shown in FIG. 18.

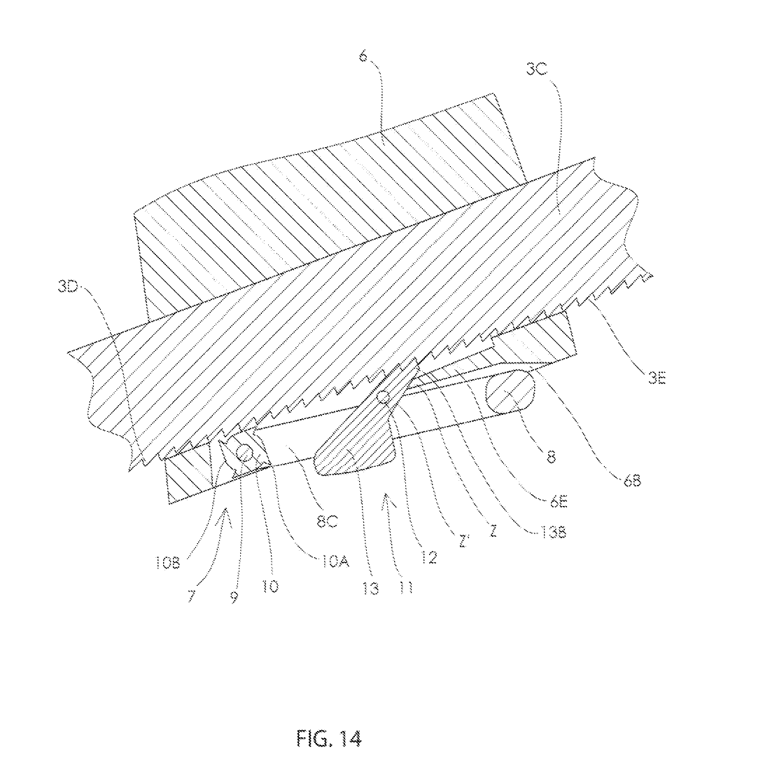

FIG. 12 is an exploded view of the retaining pawl assembly. As shown in this figure, the retaining pawl 13 comprises a retaining pawl gear tooth 13B. This retaining pawl gear tooth 13B interacts with the rack gear 3C, as shown in FIG. 14. The retaining pawl pin 12 is inserted into the retaining pawl pin hole 13A.

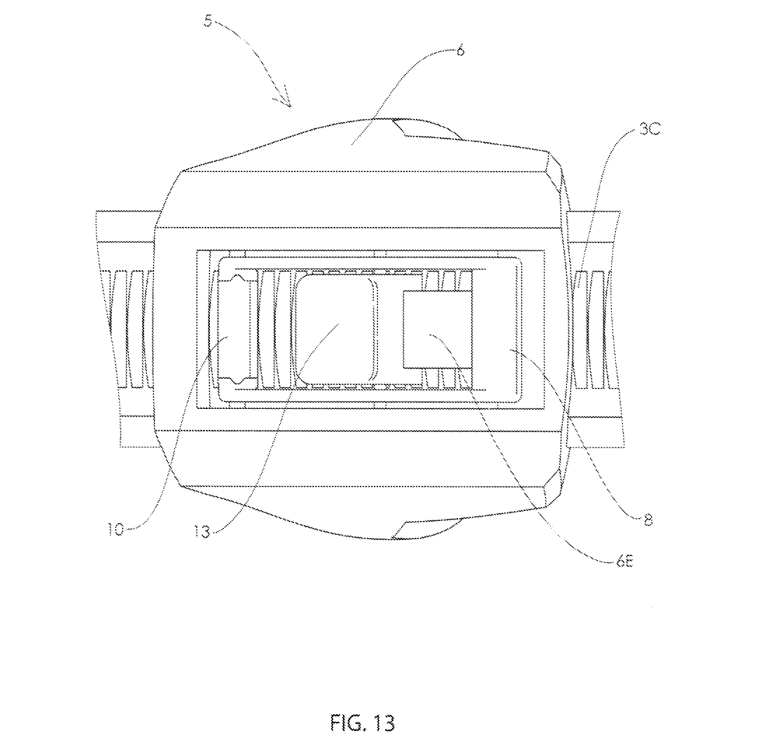

FIG. 13 is a front detail view of the adjustable, pad assembly. This figure shows the relative positions of the rack gear 3C, handle 8, advancing pawl 10 and retaining pawl 13. A retaining pawl spring 6E is situated between the handle 8 and the rack gear 3C (see also FIG. 14).

FIG. 14 a top cross-section view of the ratchet in a neutral position. As used herein, the term “ratchet” refers to the advancing lever/knob assemblies 7, 17, the retaining pawl assembly 11 or retaining pawl 18, and the rack gear 3C, 19C. As shown in this figure, the advancing pawl 10 comprises a plurality or advancing pawl gear teeth 10A. In between the advancing pawl gear teeth 10A are non-toothed sections 10B. The rack gear 3C comprises rack gear teeth 3D and rack gear recesses 3E. The advancing pawl 10 and retaining pawl spring 6E are both situated inside of the handle recess 6B, as is the retaining pawl pin 12 and a portion of the retaining pawl 13. When the ratchet is in a neutral position (i.e., no force being applied to it), the distal end (designated with “Z” in FIG. 14) of the retaining pawl spring 6E lies flat against the side surface (designated with a “Z” in FIG. 14) of the retaining paw 13. In this position, there is no compressive force on the spring 6E.

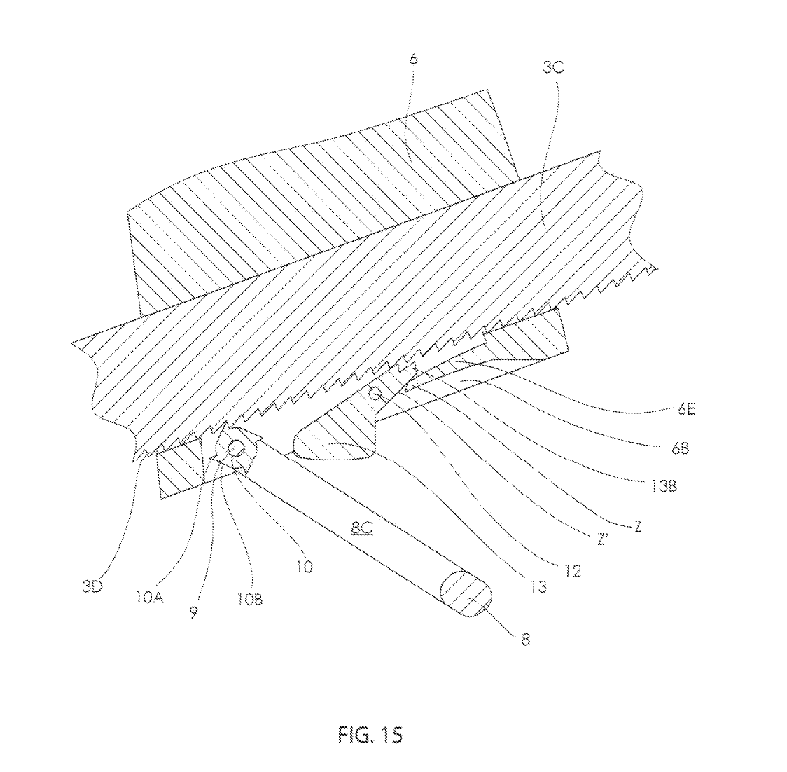

FIG. 15 is a top cross-section view of the ratchet in an advancing position. To move the position of the adjustable pad assembly 5 toward the trochanter pad 4 on the stationary pad member 3, the handle 8 is pulled outward. This movement in turn causes the advancing pawl 10 to rotate and the advancing pawl gear teeth 10A to move laterally (in FIG. 15, to the left) along the rack gear teeth 10D. As this happens, the retaining pawl gear tooth 13B is disengaged from the rack gear teeth 10D, which in turn causes the retaining pawl gear tooth 13B to push the retaining spring 6E slightly outward (i.e., toward the operator and away from the patient). As the retaining pawl spring 6E is pushed outward, compressive force is applied to the spring.

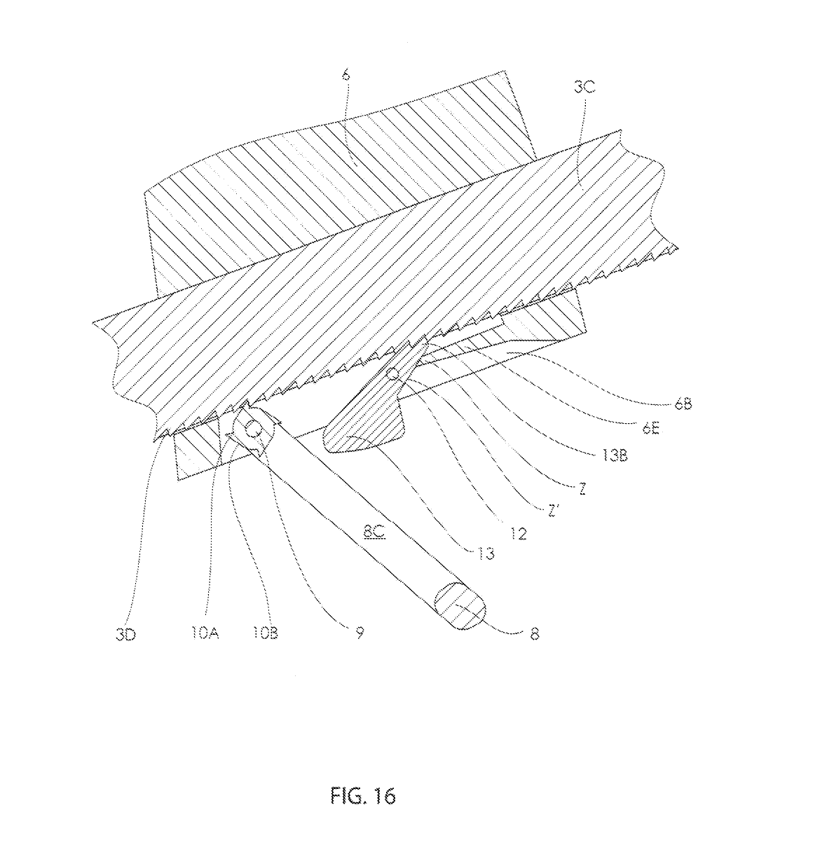

FIG. 16 is a top cross-section view of the ratchet in an advanced position. As the pawl gear tooth 13B moves laterally (i.e., to the left) along the rack gear teeth 31D, the spring 6E pushes against the side surface (Z′) of the retaining pawl 13, causing the retaining pawl gear tooth 13B to engage once again with the rack gear teeth 3D.

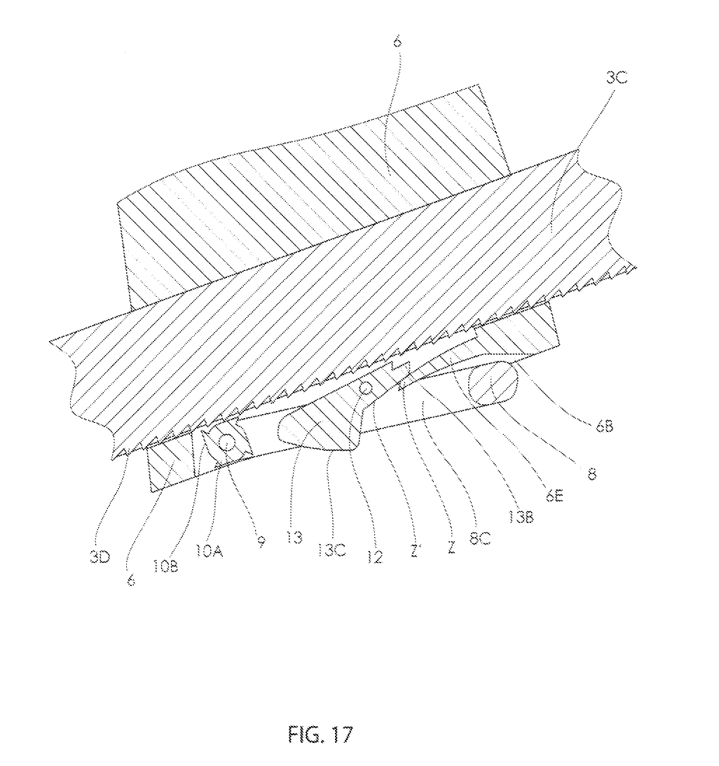

FIG. 17 is a top cross-section view of the ratchet in a releasing position. In this position, the operator has pushed on the button 13B of the retaining pawl 13. When this happens, the retaining pawl gear tooth 13B is disengaged from the rack gear teeth 3D and pushes up against the retaining pawl spring 6E. As long as the operator retains sufficient force against the spring 6E to keep it from forcing the retaining pawl gear tooth 13B to engage with the rack gear teeth 3D, the adjustably and assembly 5 can be moved in either direction (right or left) along the rack gear 3C.

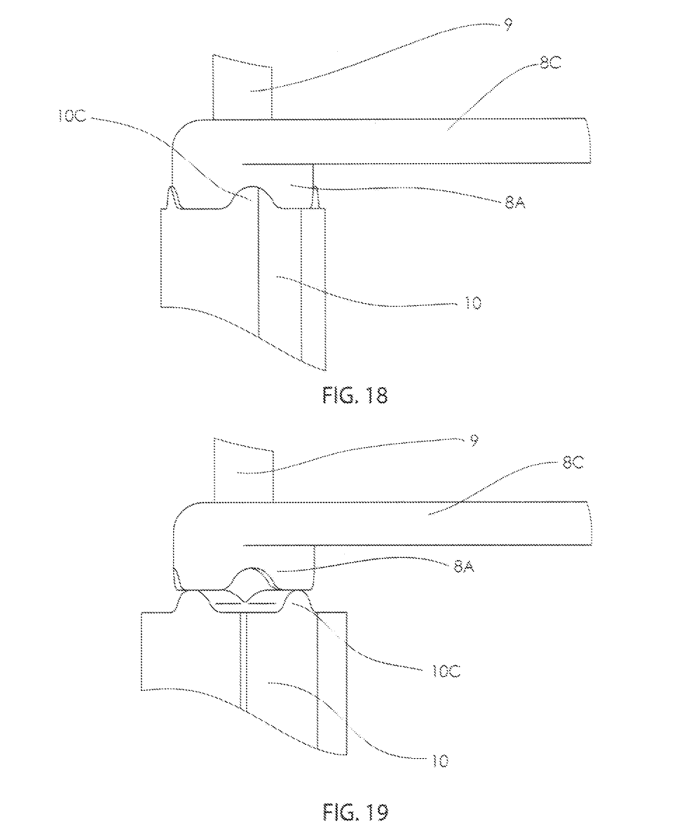

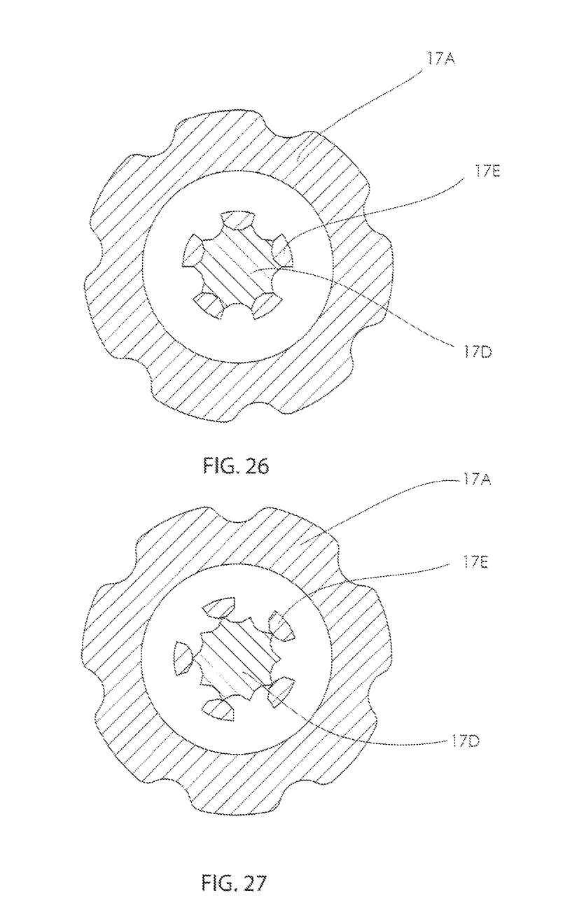

FIG. 18 is a detail view of the torque release clutch in an engaged position, and FIG. 19 is a detail view of the torque release clutch in a disengaged position. The apparatus is designed so that when a predetermined amount of pressure placed on the tore release clutch for example, because the trochanter pads are already exerting sufficient pressure on the patient), the torque release clutch will release, as shown in FIG. 19. This same principle applies to the alternate embodiment of the torque release clutch shown in FIGS. 26 and 27. These tolerances are set by the mechanical fit between the first and second faces of the torque release clutch (in the first embodiment) and the inner and outer torque release clutches (in the second embodiment).

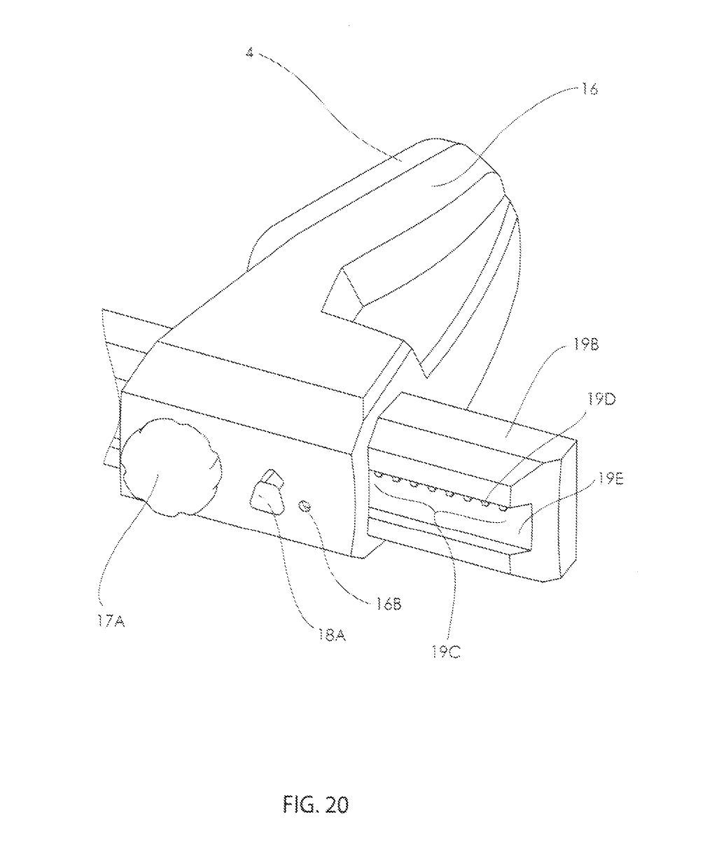

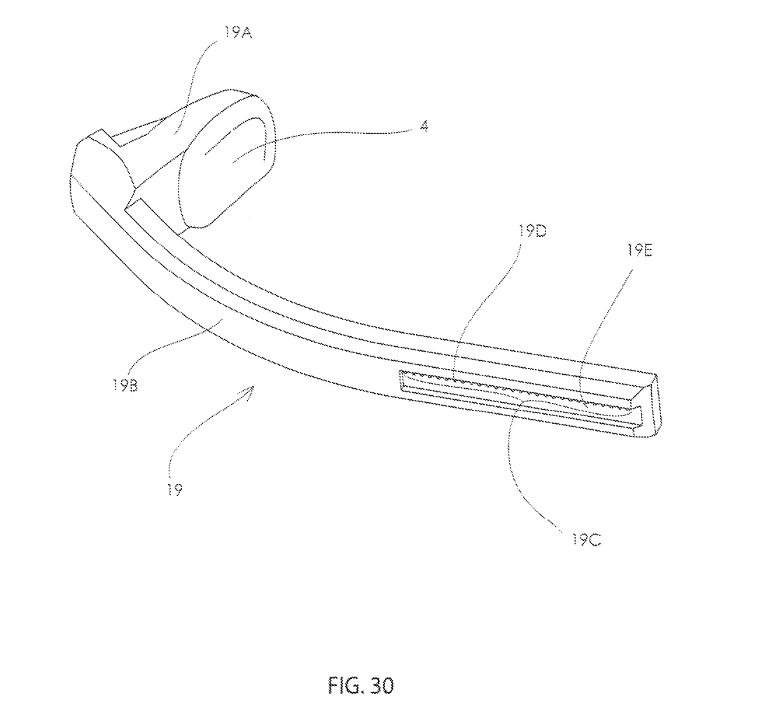

FIG. 20 is a detail view of an alternate closure and release system. This system is in lieu of the handle 8, advancing pawl 10 and retaining pawl 13 shown in FIGS. 9 and 10. In this embodiment, the cross-member 3B comprises a rack gear 3C and a rack gear recess 3E. Note that the rack gear in this embodiment is oriented differently than the rack gear of the previous embodiment in that the rack gear teeth 3D face downward rather than outward. The adjustable pad assembly 16 comprises an advancing knob assembly 17 and a retaining pawl 18. FIG. 30 shows the alternate embodiment of the stationary pad member 19 (with cross-member 19B) corresponding to the alternate closure and release system shown in FIG. 20.

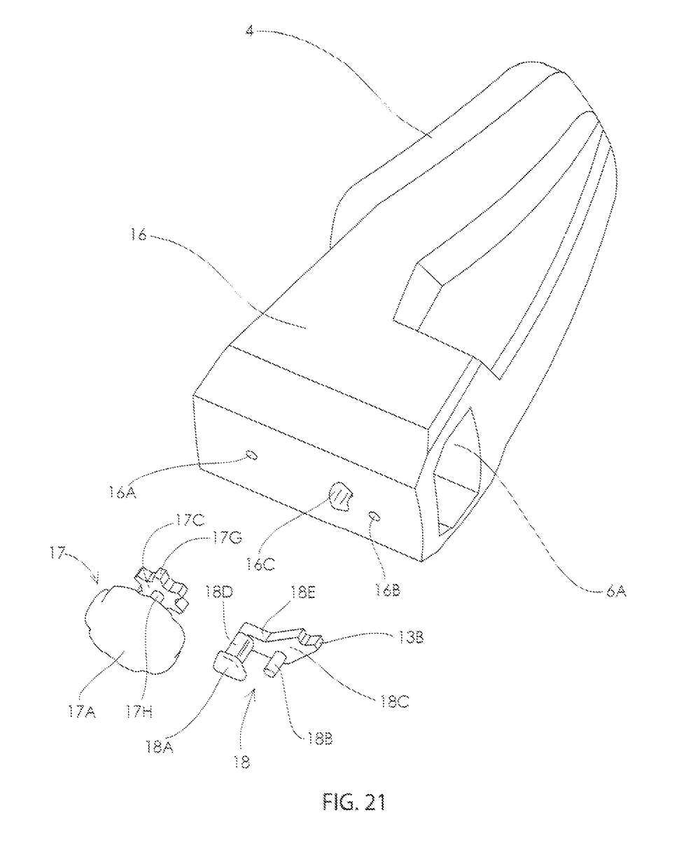

FIG. 21 is an exploded view of the alternate closure and release system. As shown in this figure, behind the knob 17A is a pinion gear 17C with pinion gear teeth 17G. The retaining pawl 18 (alternate embodiment) comprises a release aide 18A, which is attached via a release slide bracket 18D to a support member 18C that comprises a retaining pawl gear tooth 13B. A retaining pawl pin 18B extends in a forward direction from the support member 18C. When installed, the release slide bracket 18D extends through the release slide slot 16C in the front portion of the adjustable pad member 16, the retaining pawl pin 18B is inserted into the retaining pawl hole 16B, and the pinion near shaft 17H extends through the advancing knob hole 16A in the front portion of the adjustable pad member 16. In this alternate embodiment, parts 18A, 18B, 18C and 18D, collectively, are referred to as the “retaining pawl.”

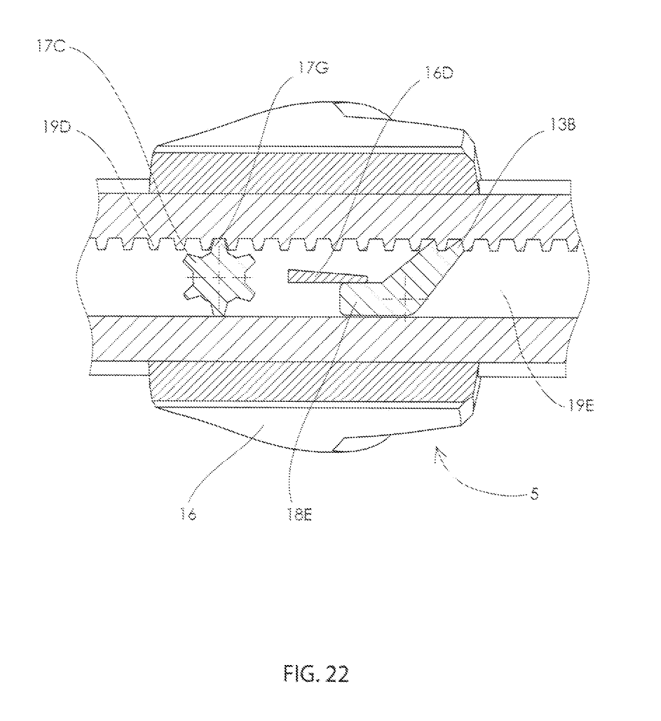

FIG. 22 is a front cross-section view of the alternate closure and release system in a neutral or advanced position. In this position, the retaining pawl spring 16D is not compressed, and the pinion gear tooth 17G is engaged with (i.e., fits into a recess in between) the rack gear teeth 19D, as is the retaining pawl gear tooth 13B.

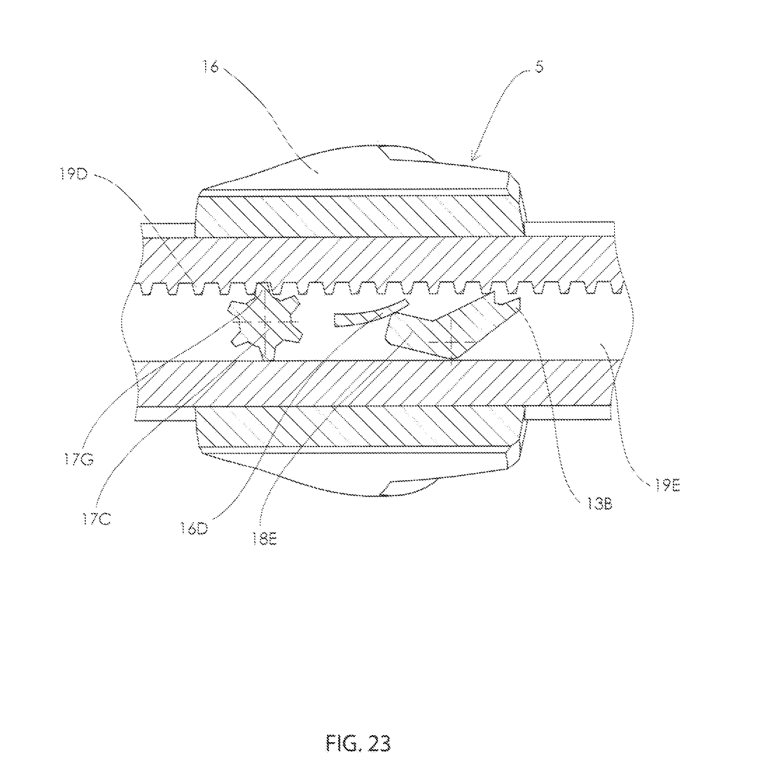

FIG. 23 is a front cross-section view of the alternate closure and release system in an advancing position. As the knob (not shown) is turned clockwise, the pinion gear 17C also turns, thereby causing the retaining pawl gear tooth 13B to become disengaged from the rack gear teeth 19D. This in turn causes the retaining pawl 18 to rotate on the retaining pawl pin 18B and the first end 18E (the end to which the release slide bracket 18D is attached) of the retaining pawl 18 to compress the retaining pawl spring 16D.

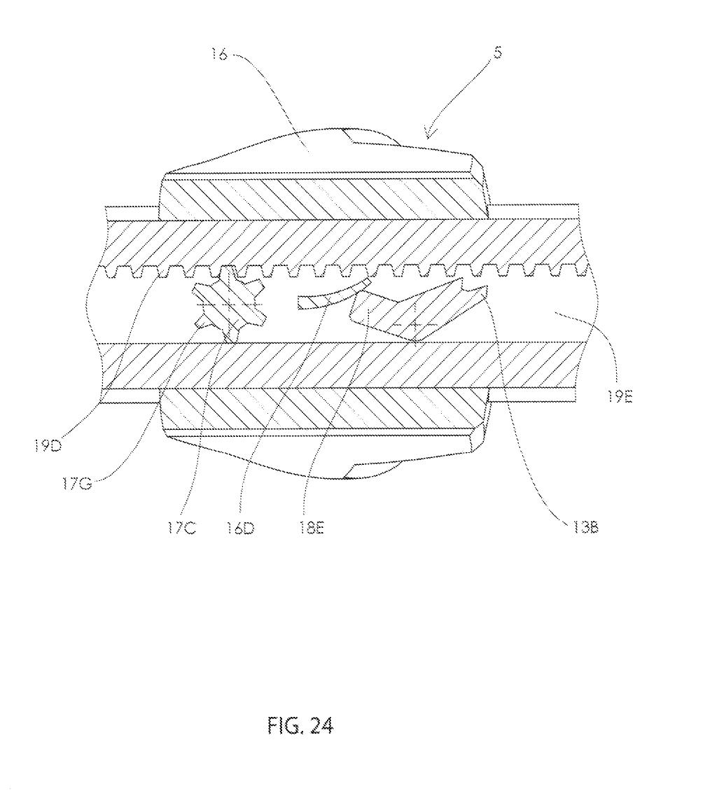

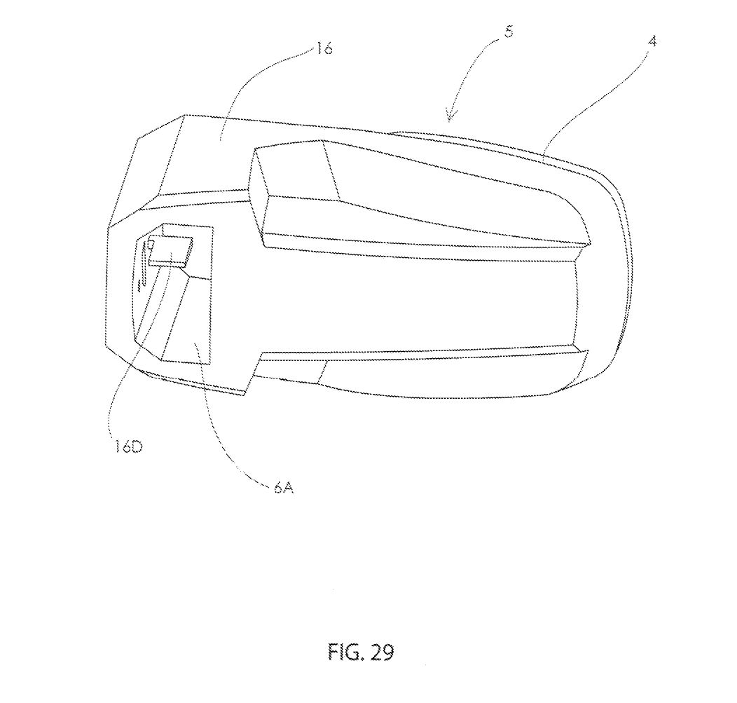

FIG. 24 is a front cross-section view of the alternate closure release system in a released position. In this position, the operator has moved the release slide 18A upward within the release slide slot 16C. This movement causes the retaining pawl gear tooth 13B to disengage from the rack gear teeth 19D and the first end 18E of the retaining pawl 18 to compress the retaining pawl spring 16D. (FIG. 29 shows how the spring 16D attaches to the adjustable pad member 16. The spring 16D may be molded to the adjustable pad member 16 or attached to the adjustable pad member in any other suitable manner, e.g., press fit.) The spring 16D puts pressure on the retaining pawl 18 to move had into an engaged position; however the retaining pawl 18 will remain in a disengaged position for as long as the operator holds the release slide 18A upward in the release slide slot 16C. In the released (or disengaged) position, the adjustable pad member 16 can slide laterally (i.e., right or left) on the cross-member 19B.

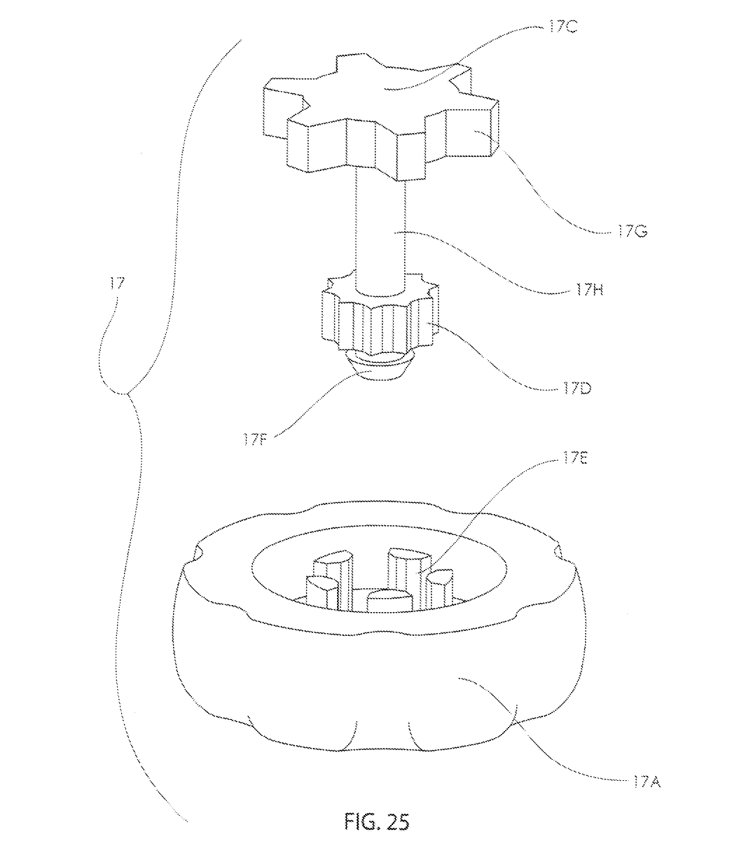

FIG. 25 is an exploded view of the knob assembly. As show in this figure, the knob assembly 17 comprises a knob 17A, pinion gear 17C, pinion gear shaft 17H, and inner torque release clutch 17D. The inner torque release &lunch 17D is situated on one end of the pinion gear shaft 17H, and the pinion gear 17C is situated on the other end of the same shaft. The inner torque release clutch 17D fits inside of circumferentially arranged protrusions on the inside surface of the knob 17A; these protrusions are referred to collectively as the outer torque release clutch 17E.

FIG. 26 is a front cross-section view of an alternate torque release clutch in an engaged position, and FIG. 27 is a front cross-section view of an alternate torque release clutch in a disengaged position. This torque release clutch embodiment works very similarly to the first torque release clutch embodiment described above; that is, when a predetermined amount of pressure is placed on the torque release clutch, the torque release clutch will release.

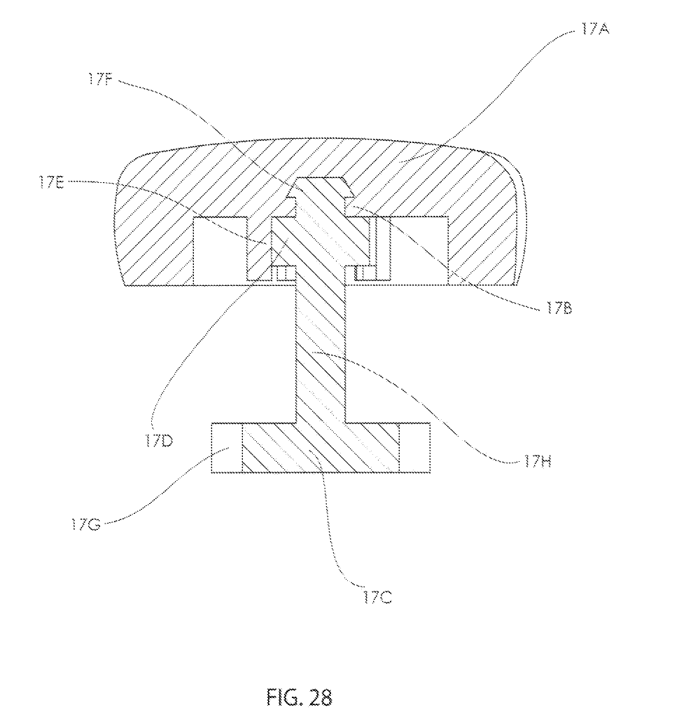

FIG. 28 is a side cross-section view of the embodiment shown in FIG. 25. All parts shown in this figure have been previously described. The present invention is not limited to any particular method of moving the adjustable pad assembly 5, 16 laterally along the cross-member, but two preferred embodiments are described herein.

Although the preferred embodiment of the present invention has been shown and described, it will be apparent to those skilled in the art that many changes and modifications may be made without departing from the invention in its broader aspects. The appended claims are therefore intended to cover all such changes and modifications as fall within the true spirit and scope of the invention.

We claim:

1. An apparatus for stabilization of pelvic fractures comprising:
(a) a stationary pad assembly comprising a stationary pad member and a first trochanter pad, the stationary pad member comprising cross-member, and the cross-member comprising a rack gear with rack gear teeth;
(b) an adjustable pad assembly comprising an adjustable pad member and a second trochanter pad;
wherein there is a distance between the first trochanter pad and the second trochanter pad, and wherein the adjustable pad assembly is configured to slide laterally on the cross-member so that the distance between the first trochanter pad and the second trochanter pad is adjustable.

2. The apparatus of claim 1, wherein each of the first and second trochanter pads is configured to fit over a greater trochanter of a human.

3. The apparatus of claim 1, wherein the adjustable pad member comprises a handle, an advancing pawl, and a retaining pawl;
wherein the cross-member fits into cross-member apertures in the adjustable pad member;
wherein the handle, advancing pawl and retaining pawl are situated in a handle recess in front of the cross-member apertures;
wherein the handle comprises a first lateral arm and a second lateral arm;
wherein an advancing pawl pin extends through a central axis if the advancing pawl, through a first end of the first lateral arm and into a first advancing pawl pin hole in a front end of the adjustable pad assembly and through a first end of the second lateral arm and into a second advancing pawl pin hole in the front end of the adjustable pad assembly;
wherein a retaining pawl pin extends through a retaining pawl pin hole in the retaining pawl and into a first retaining pawl hole in the front end of the adjustable pad assembly and a second retaining pawl hole in the front end of the adjustable pad assembly;
wherein the retaining pawl comprised a button and a retaining pawl gear tooth;
wherein when the button is pushed by an operator, the retaining pawl gear tooth disengages from the rack gear teeth of the rack gear, thereby allowing the adjustable pad member to slide laterally on the rack gear; and
wherein when the retaining pawl gear tooth is engaged with the rack gear teeth, the adjustable pad member is prevented from sliding laterally on the rack gear.

4. The apparatus of claim 3, further comprising a retaining pawl spring that is configured to bias the retaining pawl gear tooth toward engaging with the rack gear teeth.

5. The apparatus of claim 4, wherein extension of the handle cause the advancing pawl to rotate, and wherein rotation of the advancing pawl causes the retaining pawl gear tooth to disengage from the rack gear teeth until the retaining pawl spring forces the retaining pawl tooth to reengage with the rack gear teeth.

6. The apparatus of claim 1, wherein the adjustable pad member comprises a knob and retaining pawl;
wherein the knob is connected to a pinion gear via a pinion shaft;
wherein the pinion gear comprises pinion teeth;
wherein the retaining pawl comprises a release slide that is attached via a release slide bracket to a support member that comprises a retaining pawl gear tooth;
wherein the retaining pawl comprises a retaining pawl pin that extends in a forward direction from the support member and is inserted into a retaining pawl hole in a front portion of the adjustable pad member;
wherein the release slide bracket extends through a release slide in the front portion of the adjustable pad member;
wherein the pinion gear shaft extends through an advancing knob hole in the front portion of the adjustable pad member;
wherein the retaining pawl comprises a retaining pawl gear tooth;
wherein when the release slide is moved vertically within the release slot, the retaining pawl gear tooth disengages from the rack gear teeth of the rack gear, thereby allowing the adjustable pad member to slide laterally on the rack gear; and
wherein the retaining pawl tooth is engaged with the rack gear teeth, the adjustable pad member is prevented from the sliding laterally on the rack gear.

7. The apparatus if claim 6, further comprising a retaining pawl spring that is configured to bias the retaining pawl gear tooth toward engaging with the gear teeth.

8. The apparatus of claim 7, wherein rotation of the knob causes the pinion gear to rotate, and wherein rotation of the pinion gear causes the retaining pawl gear tooth to disengage from the rack gear teeth until the retaining pawl spring forces the retaining pawl gear tooth to reengage with the rack gear teeth.

9. The apparatus of claim 1, wherein the adjustable pad assembly comprises a retaining pawl with a retaining pawl gear tooth, the invention further comprising a torque release clutch that is configured to cause the retaining pawl gear tooth to disengage from the rack gear teeth when a certain force is applied to the torque release clutch.
